(19) United States
(12) Patent Application Publication
(10) Pub. No.: US 2015/0104815 A1
(43) Pub. Date: Apr. 16, 2015

(54) Porphyrin-Lipid Stabilized Nanoparticles for Surface Enhanced Raman Scattering Based Imaging

(71) Applicant: UNIVERSITY HEALTH NETWORK, Toronto (CA)

(72) Inventors: Gang Zheng, Toronto (CA); Natalie Tam, Richmond (CA)

(73) Assignee: University Health Network, Toronto (CA)

(21) Appl. No.: 14/397,097

(22) PCT Filed: Apr. 19, 2013
(86) PCT No.: PCT/CA2013/000372

§ 371 Date: Oct. 24, 2014

Related U.S. Application Data
(60) Provisional application No. 61/637,596, filed on Apr. 24, 2012.

(51) Int. Cl. G01N 21/65; G01N 33/574; G01N 33/58

(52) U.S. Cl. CPC G01N 21/658; G01N 33/587; G01N 33/57423
USPC 435/7.23; 436/164

(57) ABSTRACT

Herein are provided nano-particles comprising a nanocore of Raman-scattering material stabilized by a bilayer comprising a porphyrin-phospholipid conjugate, methods of making the same and their use in Surface Enhanced Raman Scattering.

### FIELD OF THE INVENTION

The invention relates to nanoparticles stabilized by phophyrin-lipid for use in surface enhanced Raman scattering.

### BACKGROUND OF THE INVENTION

Raman spectroscopy has expanded from molecular analysis of chemicals to molecular imaging due to its accuracy for molecular identification, photostability, and multiplexing capability¹⁻². In particular, surface enhancement Raman spectroscopy (SERS) that uses metallic nanoparticles such as gold (AuNPs), have further advanced its utility for molecular diagnostic imaging because it augments the intensity of the inelastically scattered photons up to 10¹⁴⁻¹⁵ making it ultrasensitive for detection^{1, 3}. With AuNPs, these SERS probes are inert, show low toxicity⁴, can be functionalized with targeting moieties and tuned for near infra-red (NIR) wavelengths for in vivo imaging^{3, 5}.

Currently, gold nanostructures for SERS imaging and sensing have been based on chromophores adsorbed onto its surface and subsequently encapsulated by differing surface coatings for biocompatibility and stability. The Raman dyes used commonly contain symmetrical moieties such as ones having pyrrole or benzene rings due to its strong Raman active modes with double bonds being highly polarisable⁶. Moreover, dyes are either selected for or modified to contain functional groups (e.g. thiol —SH) that allow for chemi- or physi-adsorption to metallic surfaces which may have altering affinities in the presence of differing surrounding biological matrix⁷ likely from competing thiols or oxidation⁸. To date, several different classes of surface coating, namely polyethylene glycol (PEG) and silica have emerged as stable SERS probes for in vitro and in vivo imaging^{3, 5, 9}. Recently, Applicants' group designed Raman active phospholipid gold nanoparticles (RAP AuNP), a biocompatibile and versatile alternative showing both structural and Raman signal stability using phospholipid as a surface coating¹⁰. Although it is robust, RAP AuNPs follow similar synthetic strategy as previously studied SERS probes, where the initial step of loading dye molecules on AuNPs can give rise to concentration and dye dependent inconsistencies¹¹ in Raman signals which may lead to uncontrolled aggregation and ultimately results in low reproducibility¹²⁻¹³.

### SUMMARY OF THE INVENTION

In an aspect there is provided a nanoparticle comprising a nanocore, the nanocore comprising Raman-scattering suitable material, surrounded by a bilayer comprising porphyrin-phospholipid conjugate, wherein each porphyrin-phospholipid conjugate comprises one porphyrin, porphyrin derivative or porphyrin analog covalently attached to a lipid side chain, preferably at the sn-1 or the sn-2 position, of one phospholipid.

In an aspect there is provided a method of preparing nanoparticles, comprising: preparing a solution comprising porphyrin-phospholipid conjugate, wherein the porphyrin-phospholipid conjugate comprises one porphyrin, porphyrin derivative or porphyrin analog covalently attached to a lipid side chain of one phospholipid, preferably at the sn-1 or the sn-2 position; the solution optionally further comprising other phospholipid; dehydrating the solution to provide a lipid film; and rehydrating the lipid film along with a nanocore comprising Raman-scattering suitable material; and optionally voertexing, sonicating or centrifuging the resulting solution.

In an aspect there is provided a nanoparticle produced by the method described herein.

In an aspect there is provided a method of performing Surface Enhanced Raman Scattering comprising adding the nanoparticle described herein to a sample to be analyzed and performing Surface Enhanced Raman Scattering on the sample.

In an aspect there is provided a use of the nanoparticle described herein for Surface Enhanced Raman Scattering.

In an aspect there is provided the nanoparticle described herein for use in Surface Enhanced Raman Scattering.

### BRIEF DESCRIPTION OF FIGURES

These and other features of the preferred embodiments of the invention will become more apparent in the following detailed description in which reference is made to the appended drawings wherein:

FIG. 1 shows (a) the structure of manganese pyro-lipid (MnPL) (b) and 3 step procedure for creating SERS AuNPs with MnPL.

FIG. 2 shows (a) TEM image of MnPL AuNP showing a full coverage of pyro-lipid surrounding the AuNP surface with thickness of 4-7 nm and (b) surface enhanced Raman spectrum of MnPL AuNPs with 785 nm laser (75 mW, 1 s).

FIG. 3 shows (a) normalized UV-Vis spectra of MnPL AuNPs after 24 hours in differing buffers (distilled water (ddH2O), serum, and phosphate buffered saline (PBS)) a 37° C. No change is observed for its Amax at 542 nm.

FIG. 4 shows (a) DIC and (b) Raman microscopy images of A549 lung cancer cells showing MnPL-RAP AuNP used for cellular imaging. Images were captured using 785 nm laser illumination and capturing intensity at 1239 cm-1. (c) Point spectrum measurements of MnPL AuNP on cells (green) at crosshairs of (b) vs. MnPL AuNPs in solution (black) with 785 nm laser at 3 mW integrated for 250 ms.

FIG. 5 shows (a) A549 cells that express medium levels of EGF receptor as compared to A520 cells that do not express EGF receptors, (b) dark field microscopy validating EGF receptor targeting of Pyrolipid SERS NPs. (b) MnPL nanoparticles lacking the targeting moiety penitumumab equally stain both cell lines, (c) MnPL nanoparticles with penitumumab selectively target A 549 cells expressing EGF receptor, (d) the interaction of EGFr-targeted MnPL nanoparticles can be blocked by incubating the A549 cells with 1 nM penitumumab for 30 min.

FIG. 6 shows (a) Raman microscopy illustrating EGFr targeting of pyrolipid SERS NPs linked with penitumumab to EGFr expressing A549 cells but (b) not to A520 cells that are devoid of EGFr, (c) shows that such interaction can be inhibited with pre-treatment of A520 cells with 1nM penitumumab for 30 min. (d) shows H520 cells as controls.

### DETAILED DESCRIPTION

In the following description, numerous specific details are set forth to provide a thorough understanding of the invention. However, it is understood that the invention may be practiced without these specific details.

Gold nanoparticles for surface enhanced Raman scattering (SERS) can suffer from low reproducibility due to the uncontrolled dye to gold adsorption. Porphyrins have intrinsically strong Raman scattering cross-sections, however its fluorescence properties typically overshadow its Raman detectability. Here, there is described a porphyrin-phospholipid conjugate with quenched fluorescence to serve as both Raman dye and stabilizing, biocompatible surface coating agent. We demonstrate a one-step synthesis of SERS detectable metal nanoparticle without the need for pre-adsorbed dyes. Using confocal Raman microscopy and spectroscopy, we show that this porphyrin-lipid stabilized metal nanoparticle is a novel SERS probe capable for cellular imaging. To the best of our knowledge, this is the first use of porphyrin as a Raman reporter molecule for SERS based molecular imaging.

In an aspect there is provided a nanoparticle comprising a nanocore, the nanocore comprising Raman-scattering suitable material, surrounded by a bilayer comprising porphyrin-phospholipid conjugate, wherein each porphyrin-phospholipid conjugate comprises one porphyrin, porphyrin derivative or porphyrin analog covalently attached to a lipid side chain, preferably at the sn-1 or the sn-2 position, of one phospholipid.

Raman-scattering suitable material and nanocores are known to a person skilled in the art. Examples of such materials and nanocores include Au, Ag, Cu, ZnS and Pd.

In some embodiments, a plurality of the porphyrin-phospholipid conjugate comprises a metal ion chelated therein that at least partially quenches its fluorescence. Preferably, the metal ion quenches the fluorescence of the porphyrin-phospholipid conjugate. Also preferably, the metal ion is selected from the group consisting of Cu (II), Ag (II), Mn (II/III), Co (II/III), Fe (II/III), Ni (II), Ba (II) and Cd (II), preferably Cu (II), Ag (II), Mn (II/III), Co (II/III), Fe (II/III) and Ni (II).

In some embodiments, the nanoparticle comprises in increasing preferability, 15-85 molar %, 30-70 molar %, 40-60 molar %, and about 50 molar % porphyrin-phospholipid conjugate.

In some embodiments, the porphyrin, porphyrin derivative or porphyrin analog in the porphyrin-phospholipid conjugate is selected from the group consisting of hematoporphyrin, protoporphyrin, tetraphenylporphyrin, a pyropheophorbide, a bacteriochlorophyll, chlorophyll a, a benzoporphyrin derivative, a tetrahydroxyphenyl chlorin, a purpurin, a benzochlorin, a naphthochlorins, a verdin, a rhodin, a keto chlorin, an azachlorin, a bacteriochlorin, a tolyporphyrin, a benzobacteriochlorin, an expanded porphyrin and a porphyrin isomer. Preferably, the expanded porphyrin is a texaphyrin, a sapphyrin or a hexaphyrin and the porphyrin isomer is a porphycene, an inverted porphyrin, a phthalocyanine, or a naphthalocyanine.

In some embodiments, the phospholipid in the porphyrin-phospholipid conjugate comprises phosphatidylcholine, phosphatidylethanoloamine, phosphatidylserine or phosphatidylinositol. Preferably, the phospholipid comprises an acyl side chain of 12 to 22 carbons.

In some embodiments, the porphyrin in the porphyrin-phospholipid conjugate is pyropheophorbide-a acid.

In some embodiments, the porphyrin in the porphyrin-phospholipid conjugate is a bacteriochlorophyll derivate.

In some embodiments, the phospholipid in the porphyrin-phospholipid conjugate is 1-Palmitoyl-2-Hydroxy-sn-Glycero-3-Phosphocholine or 1-Stearoyl-2-Hydroxy-sn-Gycero-3-Phosphocholine.

In some embodiments, the porphyrin-phospholipid conjugate is pyro-lipid.

In some embodiments, the porphyrin-phospholipid conjugate is oxy-bacteriochlorophyll-lipid.

In some embodiments, the porphyrin is conjugated to the glycerol group on the phospholipid by a carbon chain linker of 0 to 20 carbons.

In some embodiments, the remainder of the bilayer is comprised substantially of other phospholipid. Preferably, the other phospholipid is selected from the group consisting of selected from the group consisting of phosphatidylcholines, phosphatidylethanolamines, phosphatidic acid, phosphatidylglycerols and combinations thereof. Further preferably, the other phospholipid is selected from the group consisting of 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-dilignoceroyl-sn-glycero-3-phosphatidylcholine(DLgPC), 1,2-dipalmitoyl-sn-glycero-3-[phosphor-rac-(1-glycerol)] (DPPG), L-α-phosphatidylcholine, and combinations thereof. In one embodiment, the other phospholipid is PEGylated.

In some embodiments, the nanoparticle further comprises cholesterol.

In an aspect there is provided a method of preparing nanoparticles, comprising:

preparing a solution comprising porphyrin-phospholipid conjugate, wherein the porphyrin-phospholipid conjugate comprises one porphyrin, porphyrin derivative or porphyrin analog covalently attached to a lipid side chain of one phospholipid, preferably at the sn-1 or the sn-2 position; the solution optionally further comprising other phospholipid; dehydrating the solution to provide a lipid film; and rehydrating the lipid film along with a nanocore comprising Raman-scattering suitable material; and optionally voertexing, sonicating or centrifuging the resulting solution. Preferably, the method prepares the nanoparticle described herein.

In an aspect there is provided a nanoparticle produced by the method described herein.

In an aspect there is provided a method of performing Surface Enhanced Raman Scattering comprising adding the nanoparticle described herein to a sample to be analyzed and performing Surface Enhanced Raman Scattering on the sample.

In an aspect there is provided a use of the nanoparticle described herein for Surface Enhanced Raman Scattering.

In an aspect there is provided the nanoparticle described herein for use in Surface Enhanced Raman Scattering.

The advantages of the present invention are further illustrated by the following examples. The examples and their particular details set forth herein are presented for illustration only and should not be construed as a limitation on the claims of the present invention.

### EXAMPLE(S)

Materials and Methods

Anti-EGF Receptor MnPL SERS Nanoparticle Synthesis

Synthesis of MnPL SERS nanoparticles has been previously described. Briefly, Pyro-lipid is dissolved in methanol containing 2× molar excess of manganese chloride in the presence of pyridine and refluxed under air at 60° C. for 2 hours. MnPL is purified using solvent extraction and dried under vacuum overnight. Dry lipid film containing 100 nanomoles of MnPL, 25 nanomole DMPE, 25 namole MHPC, 50 nmole DSPE-PEG-maleimide is hydrated in the presence of 42 fmole/1 mL of citrated stabilize gold nanoparticles (60 nm) in 65° C. water bath for 30 seconds. MnPL SERS nanoparticles are washed 3× in 20 mM HEPES buffer at pH 7.4 via centrifugation (3300 rpm for 10 minutes).

Panitumumab is functionalized with reactive thiol groups using 10× molar excess of Traut's reagent at pH 8.0 for 30 minutes. Functional Panitumumab is allowed to react with MnPL SERS nanoparticles overnight at 4° C. in 20 mM HEPES buffer at PH 6.8. Sample is washed 2× in 20 mM HEPES buffer at pH 7.4 to remove free proteins and reactive salts. Particle synthesis is carried out in sterile environment to limit pyrogen contamination.

In vitro Nanoparticle Experiments

Pathogen-free, passage matched A549 and H520 human lung cancer cell lines (American Type Culture Collection) are cultured in RPMI-1640 medium with 10% fetal bovine serum and 1% penicillin and streptomycin supplement. The cell culture media is replenished every two days and cells passaged at 80% confluency.

8-well chambers are seeded with 25 000 cells per well 24 hours prior to nanoparticle incubation. Cells are fixed with 4% paraformaldehyde for 20 minutes and washed with medium. Targeted and non-targeted nanoparticles are incubated in medium containing 10% FBS at 1 pM concentration for 1 hour and washed 3× with buffer. Wells with blocked EGF receptors are incubated with 1 nmole of Panitumumab for 30 minutes prior to nanoparticle incubation.

Full spectral Raman map is acquired with a motorized Raman spectrometer coupled to a Leica DMI6000 inverted microscope containing a deep-depletion silicon CCD array with 600/1200/1800 1/mm grating and solid state excitation sources of 532, 638, and 785 nm. In vitro images are acquired with DIC image containing an overlay of hyperspectral images for a region of interest by acquiring full spectrum per point.

Dark-field microscopy is carried using an inverted microscope (Nikon TE2000) with an oil-immersion lens (100×, 0.5-1.25 NA) where oblique illumination is carried out with a dark-field stopper inside the condenser. Scattered light is only collected by the CCD detector to create the image.

Discussion

In the present study, we combined a phospholipid with a chromophore to coat AuNPs that simultaneously provides SERS detection capabilities while providing structural stability and conferring biocompatibility. In our lab, we have previously synthesized a porphyrin-lipid conjugate by linking a NIR photosensitizer, pyropheophorbide-a¹⁴⁻¹⁵, to a single acyl chain phospholipid, 16:0 lysophosphatidylcholine at the glycerol backbone. We also showed that these conjugates (pyro-lipid or PL) can self assemble into bilayer nanoparticles with phototherapy and imaging functions¹⁶⁻¹⁷. Porphyrins have strong Raman scattering owing to its heterocyclic pyrrole containing structure, though its fluorescent properties often overshadow the ability to detect its Raman spectra. Although the metal-free or closed-shell metal-inserted porphyrins are fluorescent, chelating of open-shell metal ions (e.g., Cu²⁺ or Mn³⁺) within its planar structure will quench its fluorescence. This ability to chelate divalent metallic ions on pyro-lipid was also previously demonstrated by our lab where tight packing of Cu²⁺ loaded pyro-lipid maintained bilayer stacking assemblies and could be used as PET imaging contrast agents¹⁸. On the other hand, it is known that chelating of Mn³⁺ can turn a porphyrin from a fluorescent sensor to a MRI sensor¹⁹. Therefore, insertion of suitable metal ions into porphyrins, not only, could eliminate fluorescent interference to their Raman signals, but also, introduce other imaging functions.

Thus, here we successfully used a porphyrin-lipid conjugate—one with quenched fluorescence with Mn³⁺—to, not only confer biocompatibility to AuNP surface and to stabilize AuNPs in varying aqueous buffers but also, simultaneously act as a Raman reporter. As opposed to the convention methods of using polymers (PEG), silica, or simple phospholipids to encapsulate the pre-Raman dye adsorbed gold nanoparticle, we simplified the synthesis of SERS probes by using a phospholipid-chromophore conjugate acting as the Raman reporter and protective surface coating. At the same time, we may eliminate the necessity of selecting specific dyes only suitable for adsorption to AuNPs and may provide a more reproducible strategy to obtain consistent Raman intensities when making SERS probes.

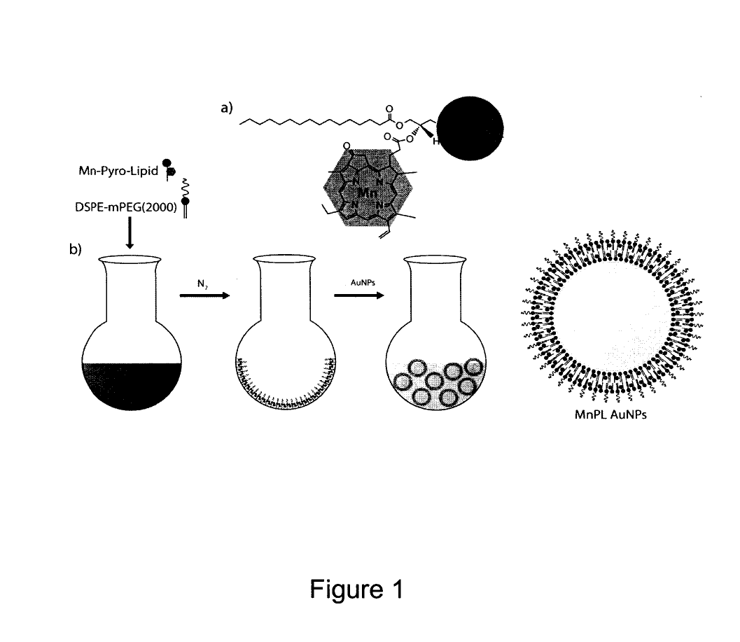

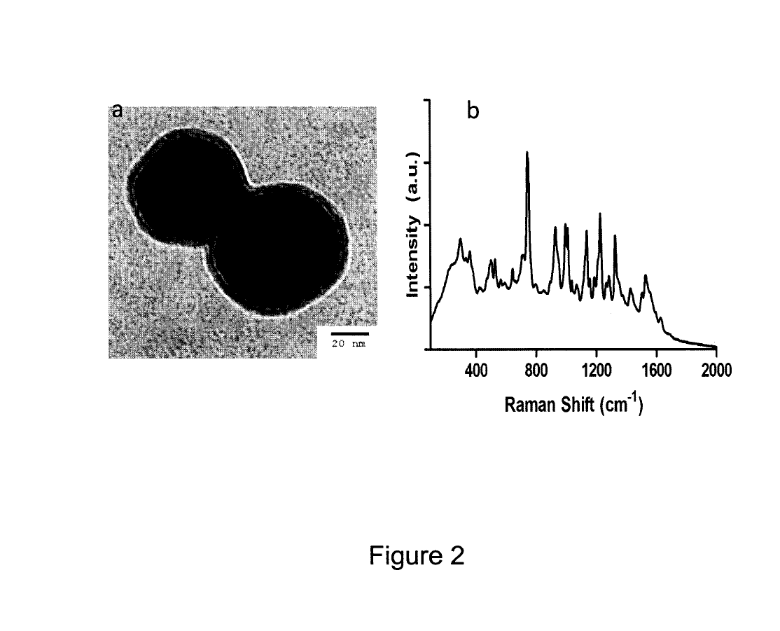

The conjugation of pyropheophorbide-a to 1-palmitoyl-2-hydroxy-sn-glycero-3-phosphocholine and the method for subsequent manganese chelation onto the pyro-lipid (MnPL) are known¹⁶. The resulting MnPL has quenched fluorescence as expected (not shown). A 1:1 ratio of MnPL is mixed with PEGylated phospholipids (1,2-distearoyl-sn-glycero-3-phosphoethanolamine-N-[methoxy(polyethylene glycol)-2000]) in chloroform and subsequently dried as a lipid film under N₂ gas in a round bottom flask (FIG. 1). The lipid film is directly hydrated with 60 nm AuNPs suspended in ddH₂O. As with creating any energetically favourable conformation with phospholipids, direct hydration often creates multilamellar vesicles; thus, the MnPL-AuNP composites are further modified by vortexing, sonication and subsequent rounds of centrifugation to ensure single bilayer coverage and any free vesicles without any entrapped gold nanoparticles are removed. The resulting structure can be seen FIG. 2 where the transmission electron microscopy (TEM) image shows a clear phospholipid coating of 4-7 nm as expected with phospholipid bilayers¹⁰.

To assess whether the MnPL coating also acts as a Raman reporter, we illuminated the purified solution using 785 nm laser to obtain its Raman spectrum (FIG. 2b). Comparing this spectrum with Raman spectra from resonance Raman scattering of similar metallo-porphyrins²⁰, each of the prominent peaks at 751 cm⁻¹, 986 cm⁻¹, 1138 cm⁻¹, 1228 cm⁻¹, 1327 cm⁻¹, 1531 cm⁻¹ were closely matched confirming that the observed spectrum displayed the pyropheophorbide-a within the nanoparticle assembly. This demonstrates that Mn³⁺ loaded pyrophorpheobide-a situated within the bilayer lipid coating is detectable by Raman spectroscopy and that it does not require the Raman dye be adsorbed on AuNP surface. Moreover, we specifically identified this as a surface enhancement effect from its interaction with AuNP surface and not from any excessive free porphyrins or pyro-lipid in solution because no Raman spectra can be detected from MnPL in solution at over 1000× without any AuNPs (not shown).

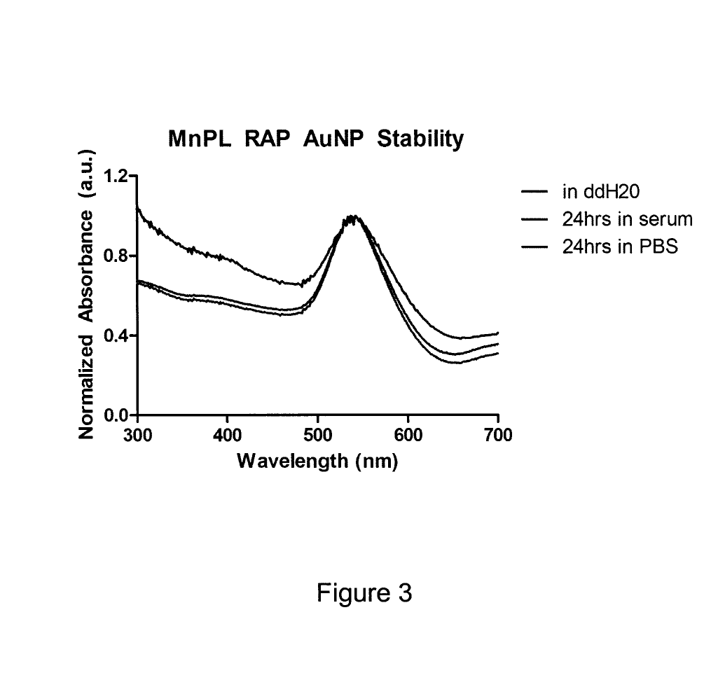

The use of MnPL as surface coating conferred outstanding biocompatible stability alike phospholipid coating alone¹⁰. As seen in FIG. 3, there is no shift in the absorption maximum (λₘₐₓ=542 nm) of MnPL AuNPs after 24 hours in either phosphate buffered saline (PBS) or 100% serum solution. The lack of any red shift of the absorption peak demonstrates that the MnPL is sufficient to prevent AuNP aggregation from serum proteins and at physiological ion concentrations. There is a broadening of the absorption peak for nanoparticles in serum which is likely due to the protein corona expected to adhere on its surface²¹⁻²².

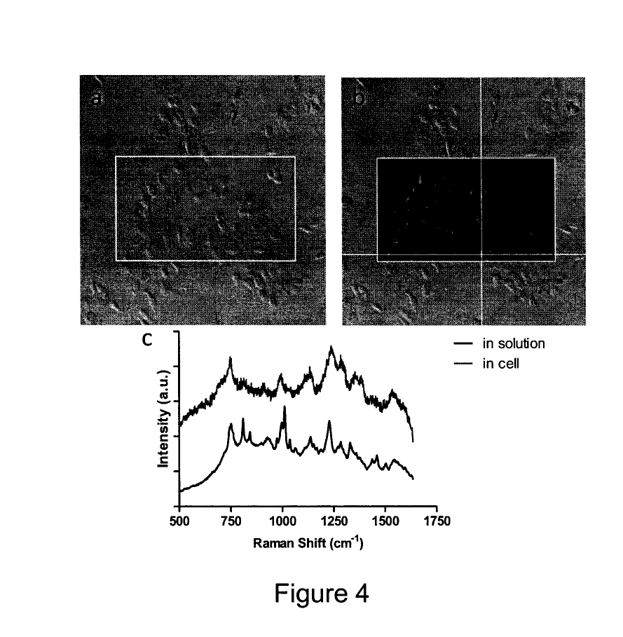

We further analyzed the MnPL AuNP as a SERS imaging probe by incubating MnPL AuNPs with A549 lung tumour cells for 1 hr at 37° C. The AuNPs were removed and cells were washed repeated with PBS and fixed with 4% paraformaldehyde. Confocal Raman microscopy maps were taken with 785 nm using raster scan mapping with integration time of 250 ms at each 5 um step. The pseudocolored image shows the signal intensity corresponding to the peak around 1239 cm⁻¹ (FIG. 4b). The DIC images (FIG. 4a) has dark spots showing clustered AuNPs on and inside the cells which align with the SERS signal detected. Although MnPL AuNPs were incubated for relatively short time, MnPL-RAP AuNPs are detected both on the periphery and inside the cells since A549 actively endocytose NPs unspecifically²³. Comparing the spectra between MnPL AuNPs in solution with the spectrum obtained within the cells, there is a both an increase in background intensity and broadening of specific peaks (FIG. 3c). The increase in background signal is likely due to the fixation and mounting reagents used to preserve cell structure for microscopy which has a weak fluorescence at 785 nm excitation. These additional molecules may also be within the SERS enhancement field leading to smaller additional peaks and broadening of the existing peaks of the MnPL AuNPs. It cannot be ruled out that the broadening of the Raman peaks may be in part from any molecular changes due aggregation or pH induced effects from endosomal uptake of the MnPL AuNPs. Nonetheless, it is clear that through the combination of porphyrin-lipid on AuNPs to create MnPL AuNPs, we are able to use porphyrins as an effective Raman reporter for SERS imaging.

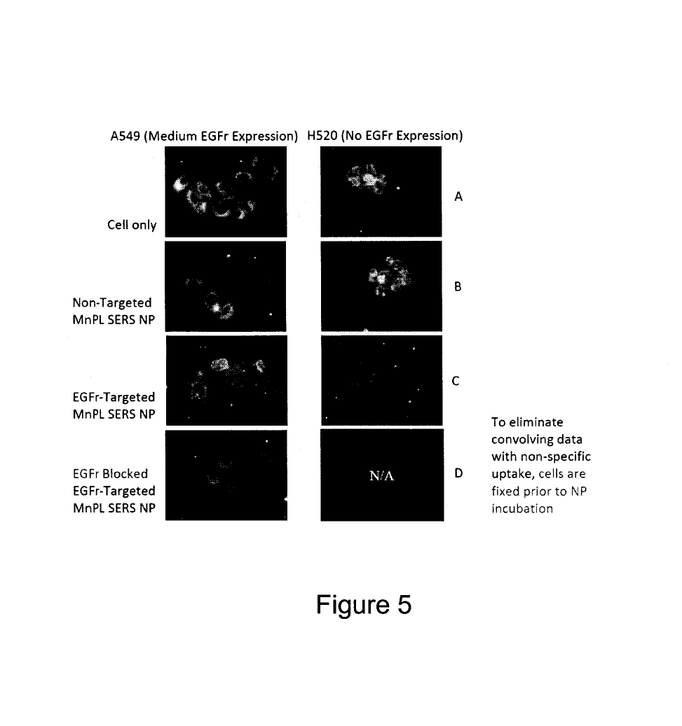

We have also examined the targeting capacity of such MnPL NP to cells that express specific target receptors. Our data (FIG. 5) indicate that MnPL NP tagged with a targeting moiety can direct the nanoparticles to those cells expressing the cognate receptor for such moiety. Data presented in FIG. 5C indicate that MnPL NP that are tagged with an antibody specific for EGF receptor (EGFr) allow for selective targeting and accumulation of such nanoparticles to cells expressing EGFr (A549 cells in FIG. 5C) and not to cells that lack the expression of EGFr (H520 cells in FIG. 5C). In these experiments soluble EGFr-specific antibody was used to demosntated that NP-antibody interaction can be inhibitied (FIG. 5D). We anticipate that Ilinking of other similar targeting moieties (peptides, proteins, aptamers, and small compounds) to such nanoparticles can allow for directing of such SERS nanoparticles to specific target cells.

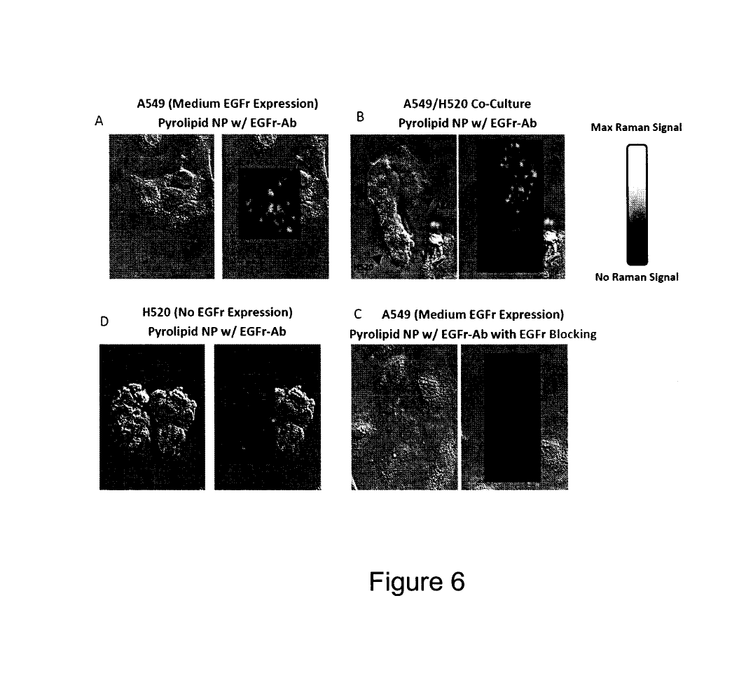

To assess whether pyrolipid SERS nanoparticles described above can distinguish cells expressing the target receptor as compared to cells that lack such receptor, we created a co-culture conditions in which EGFr expressing cells (A549—FIG. 6A) were co-cultured with cells lacking expression of such receptor (H520). Here we confimed that the MnPL nanoparticles tagged with EGFr-specific antibody (panitumumab) target A549 cells more than that compared to H520 cells (FIG. 6B). We further confirm that such targeting can be prevented by using antibody in solution prior to adding the nanoparticles (FIG. 6C).

In summary, we have demonstrated a novel strategy to create SERS probes by combining the phospholipid surface coating with a chromophore. With this approach, we expand the selection of dyes for Raman multiplexing because it no longer requires specific thiol groups for adsorption to AuNP surface. In addition, it streamlines the synthesis, provides stability, and reduces variation from the uncontrollable amount of Raman dye needed to first adsorb to gold nanostructures prior to surface functionalization. To the best of our knowledge, this is the first use of porphyrin as a

Raman reporter molecule for SERS based molecular imaging. The use of Mn-based porphyrins not only eliminates the fluorescence interference to Raman signal but also creates unique intrinsic multimodal imaging and therapy implications in addition to SERS imaging (e.g., MRI). The combination of porphyrin and phospholipid creates a highly biocompatible serum stable nanoparticle and is suited for in vivo SERS imaging where the porphyrin-lipid, derived from natural chlorophyll, is nontoxic even at 1000 mg/kg in mice¹⁶. We have further demonstrated that addition of receptor binding moieties can specifically target such nanoparticles to cells expressing those receptors.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. J. Kneipp, H. Kneipp and K. Kneipp, Chem Soc Rev, 2008, 37, 1052-1060.
2. C. L. Zavaleta, B. R. Smith, I. Walton, W. Doering, G. Davis, B. Shojaei, M. J. Natan and S. S. Gambhir, Proc Natl Acad Sci USA, 2009, 106, 13511-13516.
3. X. Qian, X. H. Peng, D. 0. Ansari, Q. Yin-Goen, G. Z. Chen, D. M. Shin, L. Yang, A. N. Young, M. D. Wang and S. Nie, Nat Biotechnol, 2008, 26, 83-90.
4. A. S. Thakor, R. Luong, R. Paulmurugan, F. I. Lin, P. Kempen, C. Zavaleta, P. Chu, T. F. Massoud, R. Sinclair and S. S. Gambhir, Sci Transl Med, 2011, 3, 79ra33.
5. J. V. Jokerst, Z. Miao, C. Zavaleta, Z. Cheng and S. S. Gambhir, Small, 2011, 7, 625-633.
6. S. Schlucker, Chemphyschem, 2009, 10, 1344-1354.
7. N. T. Flynn, T. N. T. Tran, M. J. Cima and R. Langer, Langmuir, 2003, 19, 10909-10915.
8. J. C. Love, L. A. Estroff, J. K. Kriebel, R. G. Nuzzo and G. M. Whitesides, Chem Rev, 2005, 105, 1103-1169.
9. C. L. Zavaleta, K. B. Hartman, Z. Miao, M. L. James, P. Kempen, A. S. Thakor, C. H. Nielsen, R. Sinclair, Z. Cheng and S. S. Gambhir, Small, 2011, 7, 2232-2240.
10. N. C. Tam, B. M. Scott, D. Voicu, B. C. Wilson and G. Zheng, Bioconjug Chem, 2010, 21, 2178-2182.
11. K. Faulds, R. E. Littleford, D. Graham, G. Dent and W. E. Smith, Anal Chem, 2004, 76, 592-598.
12. D. Cialla, A. Marz, R. Bohme, F. Theil, K. Weber, M. Schmitt and J. Popp, Anal Bioanal Chem, 2011.
13. C. McLaughlin, D. Graham and W. E. Smith, J Phys Chem B, 2002, 106, 5408-5412.
14. M. Zhang, Z. Zhang, D. Blessington, H. Li, T. M. Busch, V. Madrak, J. Miles, B. Chance, J. D. Glickson and G. Zheng, Bioconjug Chem, 2003, 14, 709-714.
15. J. F. Lovell, T. W. Liu, J. Chen and G. Zheng, Chemical reviews, 2010, 110, 2839-2857.
16. J. F. Lovell, C. S. Jin, E. Huynh, H. Jin, C. Kim, J. L. Rubinstein, W. C. Chan, W. Cao, L. V. Wang and G. Zheng, Nat Mater, 2011, 10, 324-332.
17. J. F. Lovell, C. S. Jin, E. Huynh, T. D. Macdonald, W. Cao and G. Zheng, Angew Chem Int Ed Engl, 2012.
18. J. Shi, T. W. Liu, J. Chen, D. Green, D. Jaffray, B. C. Wilson, F. Wang and G. Zheng, Theranostics, 2011, 1, 363-370.
19. X. A. Zhang, K. S. Lovejoy, A. Jasanoff and S. J. Lippard, Proc Natl Acad Sci U S A, 2007, 104, 10780-10785.
20. L. A. Andersson, T. M. Loehr, T. M. Cotton, D. J. Simpson and K. M. Smith, Biochim Biophys Acta, 1989, 974, 163-179.
21. M. Lundqvist, J. Stigler, G. Elia, I. Lynch, T. Cedervall and K. A. Dawson, Proc Natl Acad Sci U S A, 2008, 105, 14265-14270.
22. T. Cedervall, I. Lynch, S. Lindman, T. Berggard, E. Thulin, H. Nilsson, K. A. Dawson and S. Linse, Proc Natl Acad Sci U S A, 2007, 104, 2050-2055.
23. R. C. Stearns, J. D. Paulauskis and J. J. Godleski, Am J Respir Cell Mol Biol, 2001, 24, 108-115.

1. A nanoparticle comprising a nanocore, the nanocore comprising Raman-scattering suitable material, surrounded by a bilayer comprising porphyrin-phospholipid conjugate, wherein each porphyrin-phospholipid conjugate comprises one porphyrin, porphyrin derivative or porphyrin analog covalently attached to a lipid side chain, preferably at the sn-1 or the sn-2 position, of one phospholipid.

2. The nanoparticle of claim 1, wherein the Raman-scattering suitable material is selected from the group consisting of Au, Ag, Cu, ZnS and Pd.

3. The nanoparticle of claim 1, wherein a plurality of the porphyrin-phospholipid conjugate comprises a metal ion chelated therein that at least partially quenches its fluorescence.

4. The nanoparticle of claim 3, wherein the metal ion quenches the fluorescence of the porphyrin-phospholipid conjugate

5. The nanoparticle of claim 3, wherein the metal ion is selected from the group consisting of Cu (II), Ag (II), Mn (II/III), Co (II/III), Fe (II/III), Ni (II), Ba (II) and Cd (II), preferably Cu (II), Ag (II), Mn (II/III), Co (II/III), Fe (II/III) and Ni (II).

6. The nanoparticle of claim 1, comprising between 15-85 molar % porphyrin-phospholipid conjugate.

7. The nanoparticle of claim 1, comprising between 40-60 molar % porphyrin-phospholipid conjugate.

8. The nanoparticle of claim 1, comprising about 50 molar % porphyrin-phospholipid conjugate.

9. The nanoparticle of claim 1, wherein the porphyrin, porphyrin derivative or porphyrin analog in the porphyrin-phospholipid conjugate is selected from the group consisting of hematoporphyrin, protoporphyrin, tetraphenylporphyrin, a pyropheophorbide, a bacteriochlorophyll, chlorophyll a, a benzoporphyrin derivative, a tetrahydroxyphenyl chlorin, a purpurin, a benzochlorin, a naphthochlorins, a verdin, a rhodin, a keto chlorin, an azachlorin, a bacteriochlorin, a tolyporphyrin, a benzobacteriochlorin, an expanded porphyrin and a porphyrin isomer.

10. The nanoparticle of claim 9, wherein the expanded porphyrin is a texaphyrin, a sapphyrin or a hexaphyrin and the porphyrin isomer is a porphycene, an inverted porphyrin, a phthalocyanine, or a naphthalocyanine.

11. The nanoparticle of claim 1, wherein the phospholipid in the porphyrin-phospholipid conjugate comprises phosphatidylcholine, phosphatidylethanoloamine, phosphatidylserine or phosphatidylinositol.

12. The nanoparticle of claim 11, wherein the phospholipid comprises an acyl side chain of 12 to 22 carbons.

13. The nanoparticle of claim 1, wherein the porphyrin in the porphyrin-phospholipid conjugate is pyropheophorbide-a acid.

14. The nanoparticle of claim 1, wherein the porphyrin in the porphyrin-phospholipid conjugate is a bacteriochlorophyll derivate.

15. The nanoparticle of claim 1, wherein the phospholipid in the porphyrin-phospholipid conjugate is 1-Palmitoyl-2-Hydroxy-sn-Glycero-3-Phosphocholine or 1-Stearoyl-2-Hydroxy-sn-Gycero-3-Phosphocholine.

16. The nanoparticle of claim 1, wherein the porphyrin-phospholipid conjugate is pyro-lipid.

17. The nanoparticle of claim 1, wherein the porphyrin-phospholipid conjugate is oxy-bacteriochlorophyll-lipid.

18. The nanoparticle of claim 1, wherein the porphyrin is conjugated to the glycerol group on the phospholipid by a carbon chain linker of 0 to 20 carbons.

19. The nanoparticle of claim 1, wherein the remainder of the bilayer is comprised substantially of other phospholipid.

20. The nanoparticle of claim 19, wherein the other phospholipid is selected from the group consisting of selected from the group consisting of phosphatidylcholines, phosphatidylethanolamines, phosphatidic acid, phosphatidylglycerols and combinations thereof.

21. The nanoparticle of claim 20, wherein the other phospholipid is selected from the group consisting of 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-dilignoceroyl-sn-glycero-3-phosphatidylcholine(DLgPC), 1,2-dipalmitoyl-sn-glycero-3-[phosphor-rac-(1-glycerol)] (DPPG), L-α-phosphatidylcholine, and combinations thereof.

22. The nanoparticle of claim 20, wherein the other phospholipid is PEGylated.

23. The nanoparticle of claim 19, further comprising cholesterol.

24. A method of preparing nanoparticles, comprising:
a. preparing a solution comprising porphyrin-phospholipid conjugate, wherein the porphyrin-phospholipid conjugate comprises one porphyrin, porphyrin derivative or porphyrin analog covalently attached to a lipid side chain of one phospholipid, preferably at the sn-1 or the sn-2 position; the solution optionally further comprising other phospholipid;
b. dehydrating the solution to provide a lipid film; and
c. rehydrating the lipid film along with a nanocore comprising Raman-scattering suitable material; and optionally voertexing, sonicating or centrifuging the resulting solution.

25. The method of claim 24 for preparing the nanoparticle of claim 1.

26. A nanoparticle produced by the method of claim 24.

27. A method of performing Surface Enhanced Raman Scattering comprising adding the nanoparticle of claim 1 to a sample to be analyzed and performing Surface Enhanced Raman Scattering on the sample.

28.-29. (canceled)
